(19) United States
(12) Patent Application Publication
(10) Pub. No.: US 2022/0064597 A1
(43) Pub. Date: Mar. 3, 2022

(54) Generation of organoid-primed T (opT) cells with memory phenotype

(71) Applicant: Beth Israel Deaconess Medical Center, Inc., Boston, MA (US)

(72) Inventors: Manuel Hidalgo, Boston, MA (US); Senthil K. Muthuswamy, Boston, MA (US)

(21) Appl. No.: 17/414,907

(22) PCT Filed: Dec. 18, 2019
(86) PCT No.: PCT/US2019/067274

§ 371 Date: Jun. 16, 2021

Related U.S. Application Data
(60) Provisional application No. 62/781,440, filed on Dec. 18, 2018.

(51) Int. Cl. C12N 5/0783; A61K 35/17; G01N 33/50

(52) U.S. Cl. CPC C12N 5/0638; A61K 35/17; G01N 33/5011; G01N 33/505; C12N 2502/30; C12N 2501/2315; C12N 2501/2321; C12N 2501/115; C12N 2501/33; C12N 2501/2302

(57) ABSTRACT

A cell culture platform that uses a combination of sophisticated tissue engineering technologies to co-culture patient-derived tumor cells and the patient's own immune cells and create the conditions for expansion of tumor targeting T cells in culture. The platform can be used, e.g., for personalized medicine.

### CLAIM OF PRIORITY

This application claims the benefit of U.S. Provisional Patent Application Ser. No. 62/781,440, filed on Dec. 18, 2018. The entire contents of the foregoing are hereby incorporated by reference herein.

### TECHNICAL FIELD

Described herein is a cell culture platform that uses a combination of sophisticated tissue engineering technologies to grow patient-derived tumor cells and his/her own immune cells and create the conditions for expansion of tumor targeting T cells in culture. The platform can be used for personalized medicine.

### BACKGROUND

Advances in the understanding of cancer immunobiology and the critical role of the tumor microenvironment in facilitating immune escape and disease growth have opened a new horizon in cancer therapeutics.

### SUMMARY

The development of immunotherapeutic strategies to reverse immune paralysis, expand tumor specific effector cells, and create immune memory responses to prevent disease recurrence has begun to yield stunning clinical results. The broad applicability of this approach to other solid tumors, including pancreatic ductal adenocarcinoma and breast cancer offers significant promise to potentially prevent disease recurrence in this highly lethal disease.

Described herein are methods for preparing a co-culture. The methods include obtaining cells from a tumor in a first subject, and preparing a tumor organoid from the cells; obtaining lymphocytes from the first subject or a second subject, and suspending the lymphocytes in media comprising IL-2 (e.g., 100-10,000 u/ml), IL-15 (e.g., 1-100 ng/ml), and IL-21 (e.g., 1-100 ng/ml); and maintaining a co-culture comprising the tumor organoid (e.g., in a matrix, e.g., matrigel, fibrin gel) in the presence of the lymphocytes in media comprising IL-2, IL-15, IL-21 and polyinosinic:polycytidylic acid.

In some embodiments, preparing a tumor organoid comprises: obtaining a sample comprising tumor tissue; enzymatically digesting the tissue; plating single cell suspensions in media comprising Dulbecco's Modified Eagle Media, serum-free supplements, fibroblast growth factors (FGFs), and insulin; and incubating for 2-3 days.

In some embodiments, the first and second subjects are human.

In some embodiments, the tumor is from a pancreatic, breast, liver, or colon cancer.

In some embodiments, the methods include maintaining the co-culture comprising the tumor organoid in the presence of the lymphocytes for at least 3, 4, or 5 days.

In some embodiments, the co-culture is started at a 80:1 to 200:1 ratio, e.g., a 100:1 ratio, of effector cells to target cells, wherein the lymphocytes are effector cells, and wherein the tumor organoids are target cells.

In some embodiments, the methods include maintaining the co-culture comprising the tumor organoid in the presence of the lymphocytes for a time sufficient to produce organoid-primed, tumor targeting cytotoxic T cells (opT cells) at least 5 (e.g., 5-10) days, and optionally repeating the process two or more times to enrich for opT cells from the co-culture.

In some embodiments, the methods include administering the opT cells to the first or second subject.

In some embodiments, the opT cells are administered to the first subject from whom the tumor cells were obtained.

Also provided herein are methods for determining sensitivity of a cancer to a test compound, the method comprising: providing a co-culture as described herein; contacting the co-culture with a test compound; detecting an effect of the test compound on the co-culture by assaying for one or more of proliferation or activity of tumor-killing T cells; proliferation or activity of immune suppressive regulatory T cells, or viability or proliferation of tumor cells; and identifying a test compound that induces proliferation or activity of tumor-killing T cells, reduces proliferation or activity of immune suppressive regulatory T cells, or directly reduces viability or proliferation of tumor cells as a candidate therapeutic compound.

In some embodiments, the test compound is an immunotherapy, e.g., comprising anti-PD1, anti-PDL1 or anti-CTLA4.

In some embodiments, the methods include administering the candidate therapeutic compound to the first subject from whom the tumor cells were obtained.

Also provided herein are methods for determining tumor neo-antigens, the method comprising: providing a co-culture using a method as described herein; expanding the cells; and identifying T cell receptors expressed in the cells.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1: Exemplary schematic for application of OpT cells in a clinical setting.

FIGS. 2A-D: Expansion of peripheral blood mononuclear cells (PBMC) and priming them to kill tumor cells presented as organoids.

2A) Expansion of PBMC to tens of millions of cells in culture.

2B) Generation of organoids from the patient whose PBMC were expanded in FIG. 2A. Organoid not exposed to PBMC.

2C) Phase images of opT cells killing tumor organoids over a period of 20 hours.

2D) opT cells cultured by themselves or co-cultured with tumor organoids (1:1) for 24 hours and media analyzed for IFNg by ELISA.

FIGS. 3A-C: Characterization of resting PBMC and opT cells for activation status.

3A) opT cells have 3-fold more CD8 cells than CD4 cells compared to PBMCs where CD4 and CD8 are equally represented.

3B) CD4 positive cells in PBMC or opT cell population do not express T cell activation markers.

3C) CD8 positive cells in PBMC or opT cell population do not express T cell activation markers.

FIGS. 4A-C: opT cells respond to activation signals better than PBMC.

4A) Total population of CD4 and CD8 and CD4/CD8 double positive cells.

4B) CD4 cells in opT population respond ˜3-fold better than PBMC to activation signal by expressing IFNgamma and TNF-α (See Q2).

4C) CD8 cells in opT population respond ˜4-fold better than PBMC to activation signal by expressing IFNgamma and TNF-α (See Q2).

FIGS. 5A-B: Characterization of PBMC and opT cells for memory status.

5A) Data show a 4-fold increase in central memory cells in CD4+ opT and a modest increase in tissue-resident memory cells.

5B) Data shows a ˜7-fold increase in tissue resident memory cells in the CD8+ population and a models increase in central memory cells.

FIG. 6: Exemplary schematic for a cell-based assay for personalization of immunotherapy strategy.

FIG. 7: Exemplary schematic for the use of OpT cell platform for identification of neoantigens and its use for generation of CAR T-cells vaccines.

FIG. 8. opT cells co-cultured with tumor organoids either in the absence of presence of anti-PDL1 antibody and media analyzed for changes in IFNg levels after 72 hours; as shown the cells incubated in the presence of the anti-PDL1 antibody had increased levels of IFNg expression.

FIG. 9. opT cells, but not PBMC, are effective in entering cell cycle in response to exposure to tumor cells. PBMC or opT cells were labelled with CFSE and added to day 4 organoid cultures at the are ratio of 3:1 (T cell:tumor cell) for 72 hours before analyzing by flow cytometry. The percentage of T cells with decreased CFSE signal (a readout of cell that have completed one or more rounds of cell division) is indicated. The percentage in insert in the right column refers to the change in percentage of low CFSE cells over that observed in the absence of tumor cell coculture.

FIG. 10. opT cells are enriched for memory phenotype: CD3+/CD8+ cells were re-grouped on the basis of expression of the naïve or various T cells activation or memory phenotypes. Percentage of cells expressed associated with naïve, TRM, TRM or TM phenotypes in PBMC or opT cells are shown. Note: T cells with memory phenotype make-up >95% of the opT cells. We observed neither naïve nor exhausted T cells in the opT populations.

FIG. 11: TCRs present in opT cells retain the ability to recognize tumors when transferred to untrained T cells (B) Only the TCR that was positively selected in opT cells (DHM1), but not ones that were negatively selected-for in opT cells (DHM3), induced expression of T cell activation marker (CD69).

### DETAILED DESCRIPTION

The development of immunotherapeutic strategies to reverse immune paralysis, expand tumor specific effector cells, and create immune memory responses to prevent disease recurrence has begun to yield stunning clinical results. The applicability of this approach to cancers such as pancreatic ductal adenocarcinoma (PDAC), which are thought to lack tumor targeting T cells, represents a paradigm shift in providing a highly innovative and promising therapy to prevent disease recurrence in this highly lethal disease.

Methods of Co-Culturing Tumor Organoids and Lymphocytes

Described herein is a cell culture platform that uses a combination of sophisticated tissue engineering technologies to grow patient-derived tumor cells and his/her own immune cells and create the conditions for expansion of tumor targeting T cells in culture. A key feature of this platform is the ability to expand patient tumor cells outside the patient. The present methods include growing tumor cells (preferably tumor cells from a specific patient) as tumor organoids (mini-tumors) that retain the tumor cell phenotype in culture. The organoids are combined with patient-derived immune cells to educate and expand tumor-targeting T cells. These organoid-primed T cells (opT cells) can be re-injected then back to the patient for adoptive T cell therapy to eliminate the tumor; or used to find neoantigens; or used to personalize immune modulation approaches for patients.

Thus, described herein is a co-culture method to combine a patient's PBMC with his/her own primary tumor cells as tumor organoids (mini-tumors). These methods can be used, e.g., to provide large numbers of tumor cells that retain the tumor cell phenotype in culture, and to educate and expand tumor-targeting T cells.

The methods generally include identifying a subject who has a tumor, e.g., a cancer. As used herein, the term “cancer” refers to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. In general, a cancer will be associated with the presence of one or more tumors, i.e., abnormal cell masses. The term “tumor” is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. In general, the methods described herein can be practiced on subjects with solid tumors.

Tumors include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, cancers evaluated or treated by the methods described herein include epithelial cancers, such as a lung cancer (e.g., non-small-cell lung cancer (NSCLC)), breast cancer, colorectal cancer, kidney cancer, head and neck cancer, prostate cancer, or ovarian cancer. Epithelial malignancies are cancers that affect epithelial tissues.

Tumor Organoids

The tumor organoids used in the methods described herein can be obtained and prepared using methods known in the art. For example, the methods can include obtaining a sample comprising tumor tissue, enzymatically digesting the tissue (e.g., using collagenase) and plating single cell suspensions in a biomatrix hydrogel support, e.g., a basement membrane extract such as MATRIGEL, PATHCLEAR Grade Basement Membrane Extract (Amsbio) or other synthetic alternatives, e.g., as described in Nguyen et al., Nat Biomed Eng. 2017; 1. pii: 0096, and maintained in media containing Dulbecco's Modified Eagle Media (DMEM) with factors including serum-free supplements, fibroblast growth factors (FGFs), and insulin, e.g., the Pancreatic Progenitor and Tumor Organoid Media described in WO2016015158.

In some embodiments, the tumor cells used to grow organoids are obtained from a subject who will be treated using a method described herein; in some embodiments, the tumor cells are obtained from a different subject who has a cancer, e.g., of the same type as the subject who will be treated.

Peripheral Blood Mononuclear Cells (PBMC)

The PBMC used in the methods described herein can be obtained and prepared using methods known in the art. For example, obtain 10 ml heparinized blood from patients and centrifuge to remove plasma. The blood will be layered on top of Ficoll to separate PBMCs. PBMC will be cultured in T cell Medium Cellgro with human AB serum, IL-2, IL-15, IL-21 and Amphotericin B to generate tens of millions of PBMC.

Methods of Generating Organoid-Primed Cytotoxic T Cells (opT Cells)

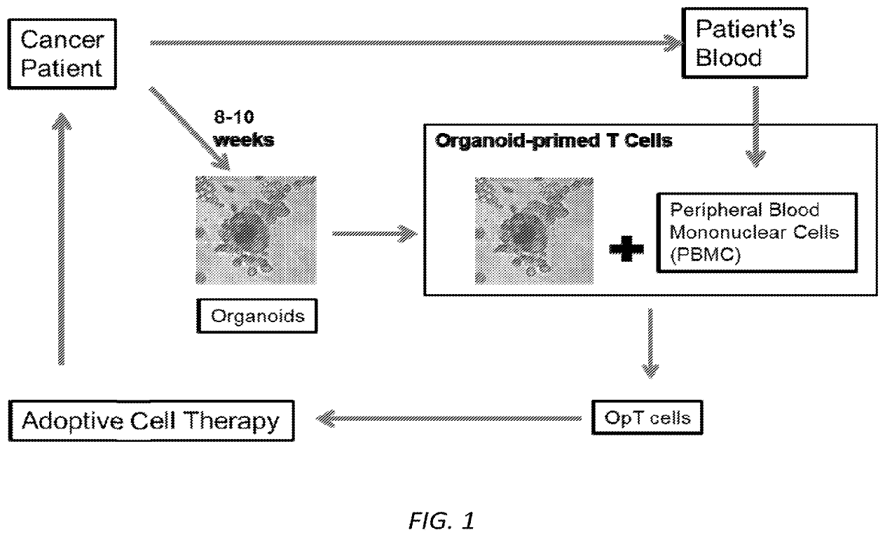

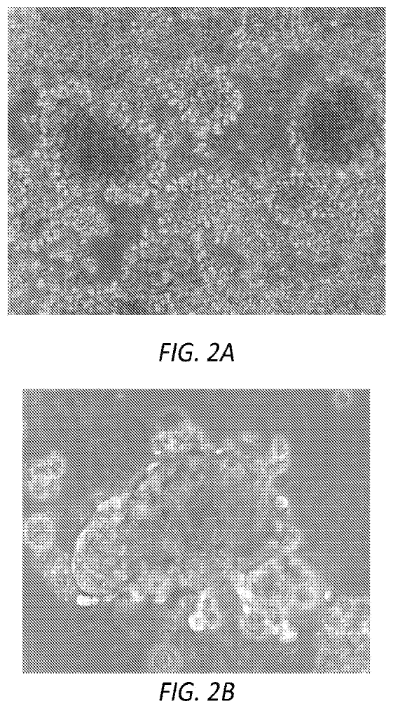

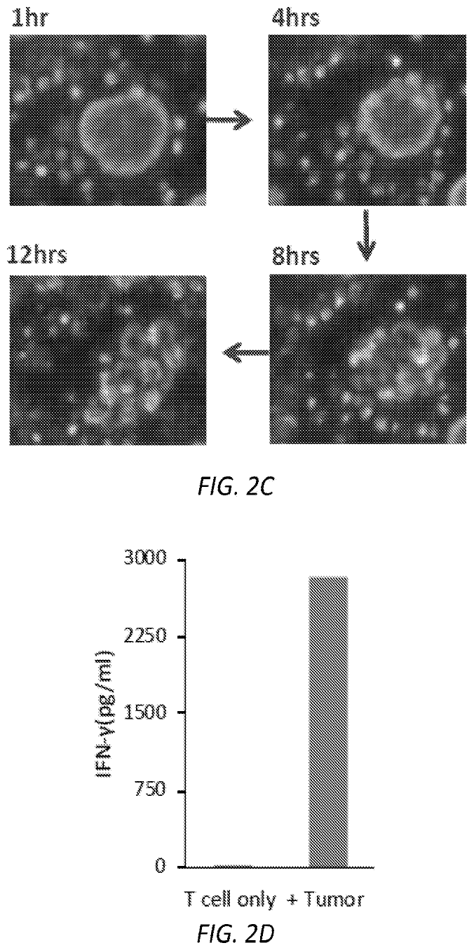

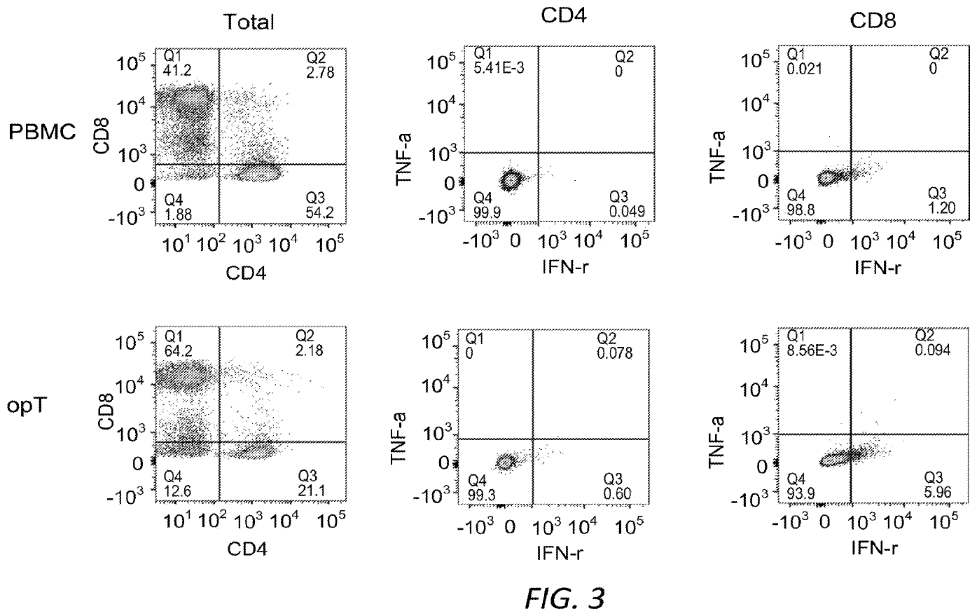

This cell-based platform methods described herein can be used, e.g., to generate organoid-primed T cells (opT cells); the CD3+ cells can be used, e.g., for adoptive cell therapy (ACT) (see Example 1, FIG. 1, FIGS. 2A-C and FIG. 3). For example, the present methods can be used to overcome the lack of presence of tumor targeting T cells with memory phenotype in cancers such as PDAC.

In adoptive cell transfer immunotherapy applications of the present methods, opT cells are isolated and re-administered back to the subject. ACT can include transfer of ex vivo expanded autologous or allogeneic tumor-reactive lymphocytes, e.g., dendritic cells or peptides with adjuvant. In some embodiments, the cells are genetically modified, e.g., to express selected T Cell Receptors and Chimeric Antigen Receptors (see, e.g., Harris et al., Trends in Pharmacological Sciences 37(3):220-230 (2016); Baruch et al., Cancer 123:2154-62 (2017)). Adoptive cell therapy protocols are known in the art, e.g., as described in Cohen et al., Immunotherapy 9(2):183-196 (2017); Redeker and Arens, Front. Immunol. 7:345 (2016). Cell populations enriched for CD3+ and memory phenotype are preferred candidates for adoptive cell therapy.

Personalized Medicine

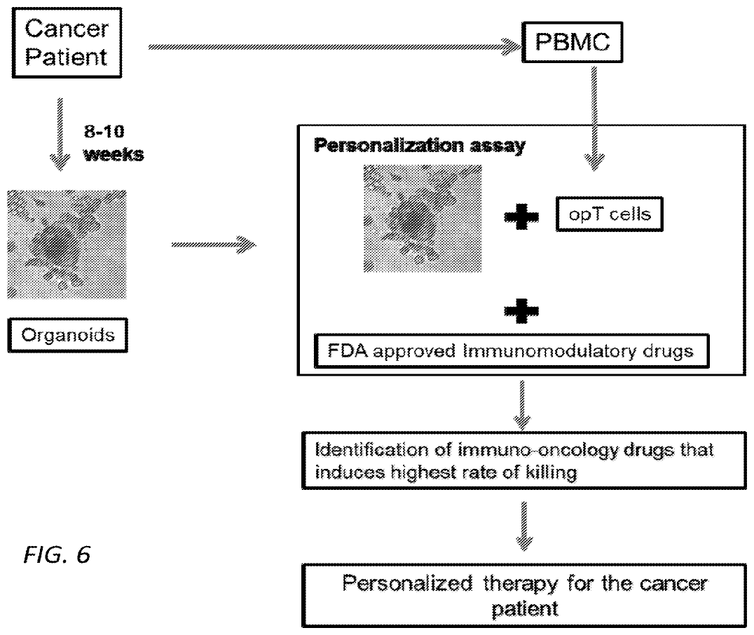

The present methods can also be used as a cell based assay platform, e.g., as a personalized test for immune oncology (FIG. 6, to identify immunomodulatory combination best treatment for each patient. In these methods, the co-culture is exposed to one or more different treatments, e.g., immunotherapies, and the ability of the lymphocytes to kill the tumor cells, e.g., the ability of the immunotherapy to induce activity of tumor-killing T cells will be evaluated by both ELISA assay for interferon gamma secretion and using intracellular flow cytometry for presence of granzyme B in CD3+ T cells. In some embodiments, once it has been determined that a treatment is effective in increasing the ability of the lymphocytes to kill the tumor cells, the treatment is administered to the subject.

In some embodiments, the immunotherapies primarily target immunoregulatory cell types such as regulatory T cells (Tregs) or M2 polarized macrophages, e.g., by reducing number, altering function, or preventing tumor localization of the immunoregulatory cell types. For example, Treg-targeted therapy includes anti-GITR monoclonal antibody (TRX518), cyclophosphamide (e.g., metronomic doses), arsenic trioxide, paclitaxel, sunitinib, oxaliplatin, PLX4720, anthracycline-based chemotherapy, Daclizumab (anti-CD25); Immunotoxin eg. Ontak (denileukin diftitox); lymphoablation (e.g., chemical or radiation lymphoablation) and agents that selectively target the VEGF-VEGFR signaling axis, such as VEGF blocking antibodies (e.g., bevacizumab), or inhibitors of VEGFR tyrosine kinase activity (e.g., lenvatinib) or ATP hydrolysis (e.g., using ectonucleotidase inhibitors, e.g., ARL67156 (6-N,N-Diethyl-D-β,γ-dibromomethyleneATP trisodium salt), 8-(4-chlorophenylthio) cAMP (pCPT-cAMP) and a related cyclic nucleotide analog (8-[4-chlorophenylthio] cGMP; pCPT-cGMP) and those described in WO 2007135195, as well as mAbs against CD73 or CD39). Docetaxel also has effects on M2 macrophages. See, e.g., Zitvogel et al., Immunity 39:74-88 (2013). In another example, M2 macrophage targeted therapy includes clodronate-liposomes (Zeisberger, et al., Br J Cancer. 95:272-281 (2006)), and M2 macrophage targeted pro-apoptotic peptides (Cieslewicz, et al., PNAS. 110(40): 15919-15924 (2013)). Immnotherapies that target Natural Killer T (NKT) cells can also be used, e.g., that support type I NKT over type II NKT (e.g., CD1d type I agonist ligands) or that inhibit the immune-suppressive functions of NKT, e.g., that antagonize TGF-beta or neutralize CD1d.

Some useful immunotherapies target the metabolic processes of immunity, and include adenosine receptor antagonists and small molecule inhibitors, e.g., istradefylline (KW-6002) and SCH-58261; indoleamine 2,3-dioxygenase (IDO) inhibitors, e.g., Small molecule inhibitors (e.g., 1-methyl-tryptophan (1MT), 1-methyl-d-tryptophan (D1MT), and Toho-1) or IDO-specific siRNAs, or natural products (e.g., Brassinin or exiguamine) (see, e.g., Munn, Front Biosci (Elite Ed). 2012 Jan. 1; 4:734-45) or monoclonal antibodies that neutralize the metabolites of IDO, e.g., mAbs against N-formyl-kynurenine.

In some embodiments, the immunotherapies may antagonize the action of cytokines and chemokines such as IL-10, TGF-beta, IL-6, CCL2 and others that are associated with immunosuppression in cancer. For example, TGF-beta neutralizing therapies include anti-TGF-beta antibodies (e.g., fresolimumab, Infliximab, Lerdelimumab, or GC-1008), antisense oligodeoxynucleotides (e.g., Trabedersen), and small molecule inhibitors of TGF-beta (e.g. LY2157299), (Wojtowicz-Praga, Invest New Drugs. 21(1): 21-32 (2003)). Another example of therapies that antagonize immunosuppression cytokines can include anti-IL-6 antibodies (e.g. siltuximab) (Guo, et al., Cancer Treat Rev. 38(7):904-910 (2012)). mAbs against IL-10 or its receptor can also be used, e.g., humanized versions of those described in Llorente et al., Arthritis & Rheumatism, 43(8): 1790-1800, 2000 (anti-IL-10 mAb), or Newton et al., Clin Exp Immunol. 2014 July; 177(1):261-8 (Anti-interleukin-10R1 monoclonal antibody). mAbs against CCL2 or its receptors can also be used. In some embodiments, the cytokine immunotherapy is combined with a commonly used chemotherapeutic agent (e.g., gemcitabine, docetaxel, cisplatin, or tamoxifen) as described in U.S. Pat. No. 8,476,246.

In some embodiments, immunotherapies can include agents that are believed to elicit “danger” signals, e.g., “PAMPs” (pathogen-associated molecular patterns) or “DAMPs” (damage-associated molecular patterns) that stimulate an immune response against the cancer. See, e.g., Pradeu and Cooper, Front Immunol. 2012, 3:287; Escamilla-Tilch et al., Immunol Cell Biol. 2013 November-December; 91(10):601-10. In some embodiments, immunotherapies can agonize toll-like receptors (TLRs) to stimulate an immune response. For example, TLR agonists include vaccine adjuvants (e.g., 3M-052) and small molecules (e.g., Imiquimod, muramyl dipeptide, CpG, and mifamurtide (muramyl tripeptide)) as well as polysaccharide krestin and endotoxin). See Galluzi et al., Oncoimmunol. 1(5): 699-716 (2012), Lu et al., Clin Cancer Res. Jan. 1, 2011; 17(1): 67-76, U.S. Pat. Nos. 8,795,678 and 8,790,655. In some embodiments, immunotherapies can involve administration of cytokines that elicit an anti-cancer immune response, see Lee & Margolin, Cancers. 3: 3856-3893(2011). For example, the cytokine IL-12 can be administered (Portielje, et al., Cancer Immunol Immunother. 52: 133-144 (2003)) or as gene therapy (Melero, et al., Trends Immunol. 22(3): 113-115 (2001)). In another example, interferons (IFNs), e.g., IFNgamma, can be administered as adjuvant therapy (Dunn et al., Nat Rev Immunol. 6: 836-848 (2006)).

In some embodiments, immunotherapies can antagonize cell surface receptors to enhance the anti-cancer immune response. For example, antagonistic monoclonal antibodies that boost the anti-cancer immune response can include antibodies that target CTLA-4 (ipilimumab, see Tarhini and Iqbal, Onco Targets Ther. 3:15-25 (2010) and U.S. Pat. No. 7,741,345 or Tremelimumab) or antibodies that target PD-1 (nivolumab, see Topalian, et al., NEJM. 366(26): 2443-2454 (2012) and WO2013/173223A1, pembrolizumab/MK-3475, Pidilizumab (CT-011)).

Some immunotherapies enhance T cell recruitment to the tumor site (such as Endothelin receptor-A/B (ETRA/B) blockade, e.g., with macitentan or the combination of the ETRA and ETRB antagonists BQ123 and BQ788, see Coffman et al., Cancer Biol Ther. 2013 February; 14(2):184-92), or enhance CD8 T-cell memory cell formation (e.g., using rapamycin and metformin, see, e.g., Pearce et al., Nature. 2009 Jul. 2; 460(7251):103-7; Mineharu et al., Mol Cancer Ther. 2014 Sep. 25. pii: molcanther.0400.2014; and Berezhnoy et al., Oncoimmunology. 2014 May 14; 3:e28811). Immunotherapies can also include administering one or more of: cytokines (e.g., IL-2), cyclophosphamide, anti-interleukin-2R immunotoxins, Prostaglandin E2 Inhibitors (e.g., using SC-50) and/or checkpoint inhibitors including antibodies such as anti-CD137 (BMS-663513), anti-PD1 (e.g., Nivolumab, pembrolizumab/MK-3475, Pidilizumab (CT-011)), anti-PDL1 (e.g., BMS-936559, MPDL3280A), or anti-CTLA-4 (e.g., ipilumimab; see, e.g., Kruger et al., “Immune based therapies in cancer,” Histol Histopathol. 2007 June; 22(6):687-96; Eggermont et al., “Anti-CTLA-4 antibody adjuvant therapy in melanoma,” Semin Oncol. 2010 to October; 37(5):455-9; Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August; 33(6):570-90; Moschella et al., “Combination strategies for enhancing the efficacy of immunotherapy in cancer patients,” Ann N Y Acad Sci. 2010 April; 1194:169-78; Ganesan and Bakhshi, “Systemic therapy for melanoma,” Natl Med J India. 2010 January-February; 23(1):21-7; Golovina and Vonderheide, “Regulatory T cells: overcoming suppression of T-cell immunity,” Cancer J. 2010 July-August; 16(4):342-7.

Cancer Vaccines

Cancer vaccine approaches need large amounts of tumor cells to generate dentritic cell-tumor cell fusions. Unlike hematopoietic malignancies where it is relatively easy to have access to large quantities of tumor cells, carcinomas pose a challenge to have access to large quantities of tumor cells. The present methods can be used to produce large numbers of patient-derived tumor cells, as the present methodology overcomes a long-standing bottleneck of keeping patient tumor cells alive in culture and maintaining its tumor traits long-enough to use to educate T cells. Tumor cells generated from the organoids cultured as described herein can be fused with autologous dendritic cells (DCs) to generated cancer vaccines, e.g., DC-tumor fusion cells (DC-tumor FCs) as described in Koido, Int J Mol Sci. 2016 June; 17(6): 828; Koido and Gong, Methods Mol Biol. 2015; 1313:185-91; and Takakura et al., Discov Med. 2015 March; 19(104):169-74. The fusion cells are then re-injected back in to the patient to elicit an immune response.

Identifying Cancer Neo-Antigens

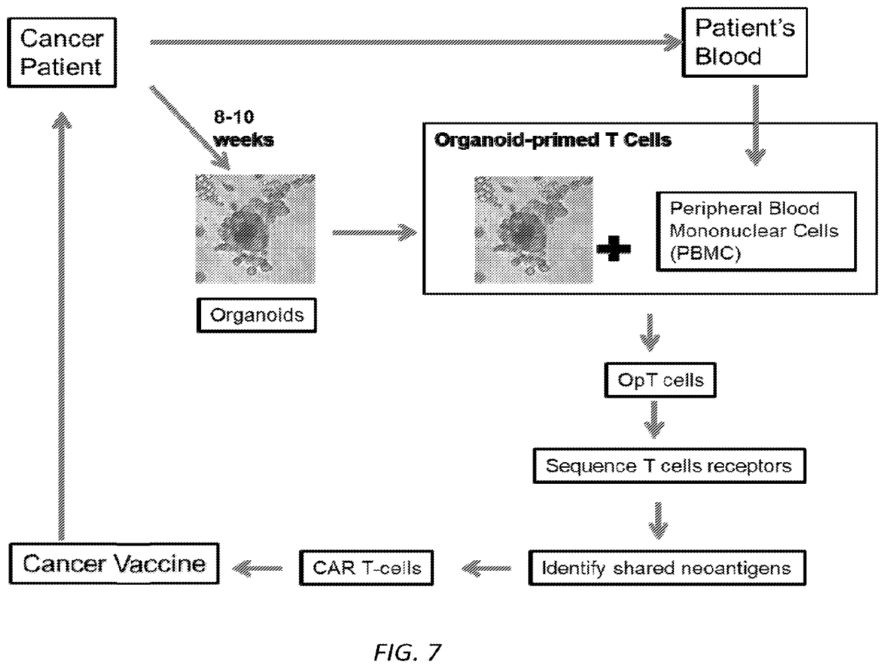

The present methods also provide a platform to identify functional tumor neo-antigens, which can be used, e.g., for design of new CAR T-cell vaccines, e.g., as shown in FIG. 7. The methods can include isolating opT cells from the co-culture, expanding the cells, and subjecting them to T cell receptor (TCR) sequencing. T cell clones that are enriched during repeated stimulation and most abundantly represented in each opT population and shared among multiple opT cell population are used to design CAR receptors, which will used for engineering T cells for adoptive cell therapy.

Methods of Screening

The present methods also provide a cell-based discovery platform for use in identifying new agents that can enhance anti-tumor immune response; these methods include exposing the co-culture to test compounds, and determining what effect the compound has on the lymphocytes or tumor organoid in the co-culture.

These methods can be used for screening test compounds, e.g., polypeptides, polynucleotides, inorganic or organic large or small molecule test compounds, to identify agents useful in the treatment of cancers, e.g., new immunotherapies.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, e.g., Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the present methods can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample comprising a co-culture, and one or more effects of the test compound is evaluated. For example, the ability of the test compound to enhance activity of T cells will be evaluated by both ELISA assay for interferon gamma secretion and using intracellular flow cytometry for presence of granzyme B in CD3+ T cells.

A test compound that has been screened by a method described herein and determined to induce proliferation or activity of tumor-killing T cells, to reduce proliferation or activity of immune suppressive regulatory T cells, or to directly reduce viability or proliferation of tumor cells, can be considered a candidate compound. A candidate compound that has been screened, e.g., in an in vivo model of a cancer, e.g., a xenograft model, and determined to have a desirable effect on the disorder, e.g., on one or more symptoms of the disorder (e.g., tumor size, number, or metastasis), can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that induce proliferation or activity of tumor-killing T cells, to reduce proliferation or activity of immune suppressive regulatory T cells, or to directly reduce viability or proliferation of tumor cells) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating cancer, e.g., carcinomas, e.g., breast, liver, pancreatic, or colon cancer. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a cancer. The animal can be monitored for a change in the disorder, e.g., for an improvement in a parameter of the disorder, e.g., a parameter related to clinical outcome. In some embodiments, the parameter is tumor size or growth, and an improvement would be a reduction in tumor size or growth rate. In some embodiments, the subject is a human, e.g., a human with cancer, and the parameter is tumor size or growth, recurrence or metastasis.

### EXAMPLES

The invention is further described in the following examples, which do not limit the scope of the invention described in the claims.

Materials and Methods

The following materials and methods were used in the Examples below.

Digestion Media:

DMEM

1:100 dilution of 100 mg/ml stock Collagenase/Dispase

1.0% Penicillin-streptomycin

Resuspension Media: DMEM

1.0% Penicillin-streptomycin

1.0% BSA

Add 1% BSA by weight to DMEM+1.0% Pencillin-streptomycin containing media and stir to dissolve the BSA. Once dissolved, filter sterilize using 0.2μ filter and store at 4° C.

PaTOM Growth Media:

DMEM: 500 ml

5.0 ml of PaTOM Growth factors cocktail* (Tube A) *Contains: B27, ascorbic acid, insulin, hydrocortisone, FGF2, and all-trans retinoic acid

1.0% Penicillin-streptomycin

250 μl Hydrocortizone (1.0 mg/ml) (Tube B)

Culture Media:

PaTOM growth media

5.0% GFR-Matrigel

10 μM Y267632, Rock inhibitor (10 mM, a 1000× stock). Rock inhibitor is prepared in sterile Phosphate Buffered Saline.

Freezing Media:

Cryostar freezing media

10 μM Y267632, Rock inhibitor

### Example 1. Generating Organoid-Primed, Tumor Targeting Cytotoxic T Cells (opT Cells)

Human Pancreas ductal adenocarcinoma was used to generate organoid cultures using Tumor Organoid Media and methodology described in WO2016015158 and above (see, e.g., FIG. 2B).

In one exemplary method, tumor/biopsy tissue was placed in Resuspension media, minced, and pelleted by centrifugation at 1500 rpm for 5 min at 4° C. The tissue pellet was resuspended in Digestion media for 15-30 minutes or so until digested. Additional resuspension media was then used to transfer to a tube for centrifugation to pellet. Accutase was mixed into the pellet and the cells were incubated at 37° C. for 30 min before resuspension, purification with a tissue strainer to remove cell debris, and centrifugation. The pelleted cells were resuspended in fresh Culture Media with matrigel, and transferred in single droplets into wells of a matrigel-coated 12-well dish.

For PBMC expansion from peripheral blood, 10 ml heparin blood from patients was centrifuged and plasma was separated. Blood was layered on top of Ficoll (GE) bed in a 15 ml tube. After centrifugation, the PBMCs were transferred to a new 15 ml tube add centrifuged. The supernatant was discarded, and PBMC were plated in medium (Cellgro with 10% human AB serum, containing IL-2 (1000 u/ml), IL-15 (10 ng/ml), IL-21 (10 ng/ml), and Amphotericin B (FIG. 2A).

Co-Culture with Organoids

60,000 T cells and 60,000 tumor cells were cultured in one well (96 well plate, U bottom) with 200 ul T cell medium. One week later, three wells of T cells were combined into one well (24 well plate). In some embodiments,

Tumor cells were added again at a 1:1 ratio; in some cases the process was repeated 3 times to enrich for opT cells that are effective at killing tumor cell derived organoids. The culture muedia included IL2 to support growth and viability of T cells, but did not have the sufficient growth factors to support the myeloid and B cells lineages.

Live imaging analysis of labelled tumor organoid co-cultured with unlabeled opT cells showed efficient killing of organoids within 24 hours of co-culture (FIG. 2C). Furthermore, T cells that were recovered from the co-culture had the ability to kill a new batch organoids in 48-72 hours suggesting that the co-culture primed the T cells to acquire cytotoxic killing activity. We referred to these cells as organoid-primed T (opT) cells. As shown in FIG. 2D, opT cells co-cultured with tumor organoids (1:1) for 24 hours and media analyzed for IFNg by ELISA.

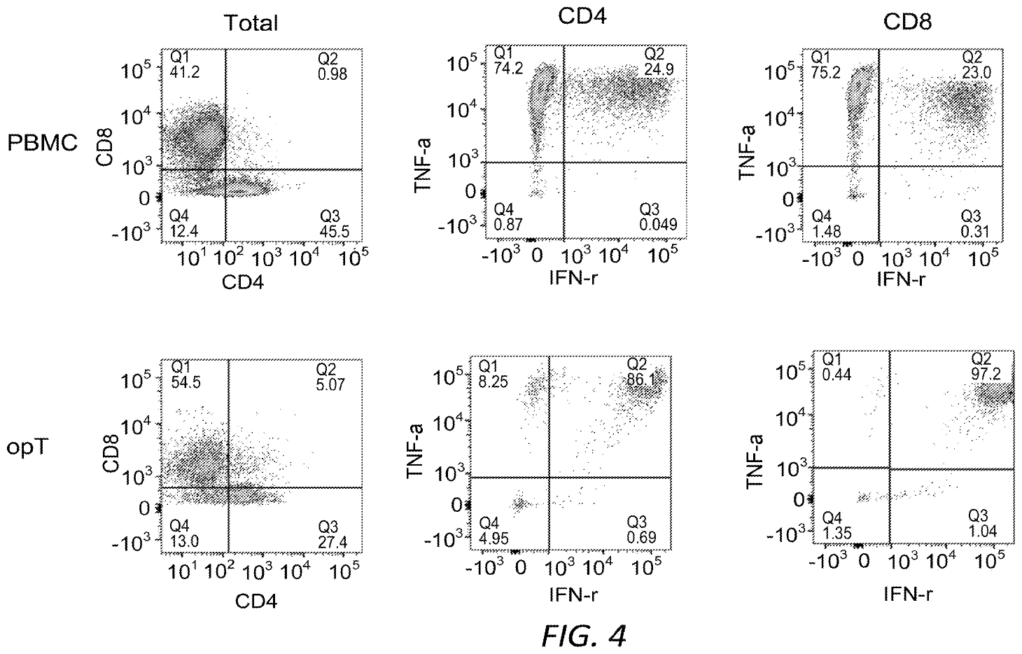

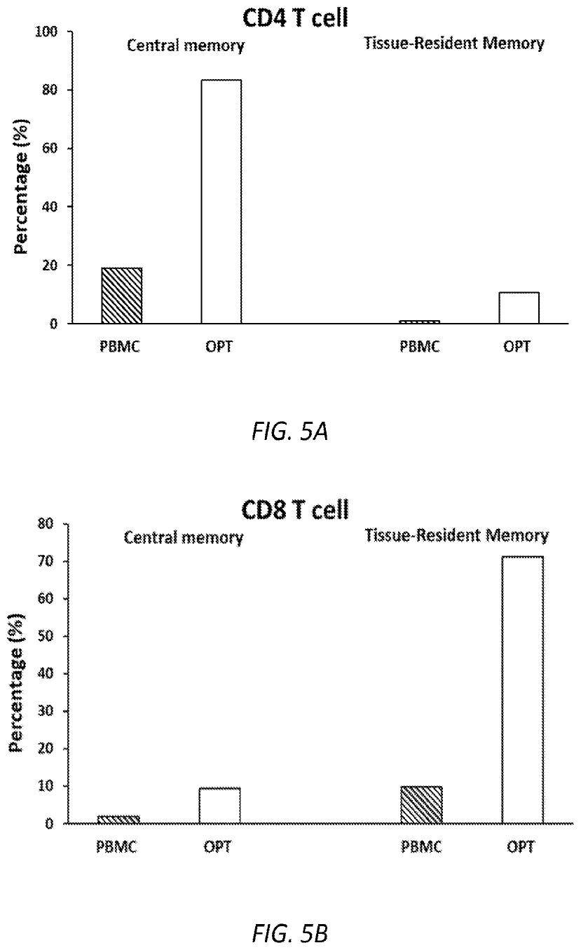

The opT cells included both CD4+ and CD8+ T cells (FIG. 3A), as observed in PBMC. Unstimulated CD4+ and CD8+ PBMC or opT cells did not express activation marker interferon-gamma and TNFa (FIG. 3B). Activated PBMC or opT cells represented CD4+ and CD8+ populations (FIG. 4A).

Activated CD4+ and CD8+ opT showed a 3-fold increase in expression activation markers, TNFa and IFNg (FIG. 4B, C).

Activated CD+ opT cells showed a 4-fold increase in central memory cells (CD45RA−; CCR7+) and, activated CD8+ opT cells show a 4-fold increase in tissue-resident memory marker (CD103+).

### Example 2. Tumor Organoid-T Cell Co-Culture as a Platform to Understand Immune Modulation

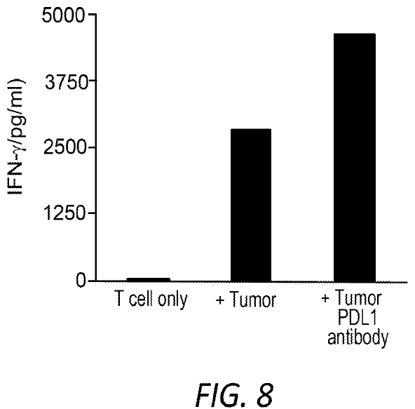

We investigated whether this co-culture platform could provide insights in immune-modulatory molecules that are active. Interestingly the tumor organoids were positive for expression of PDL1. Consistent with this observation, addition of anti-PDL1 antibody to the tumor organoid:opT cell co-culture induced an increase in IFNg secretion (FIG. 8), demonstrating that the co-culture platform can be used for evaluating the role played by immune checkpoint regulators. Although this platform lacks the tumor microenvironment it can serve as an in vitro platform to determine whether a given immunomodulatory drug will be effective for a specific subject, providing a personalized test for immune oncology.

### Example 3. A Lab-Based Platform for Expanding Tumor Targeting

Cytotoxic T Cells for Adoptive T Cells Therapy.

opT Cells Respond to Organoids by Entering Cell Cycle.

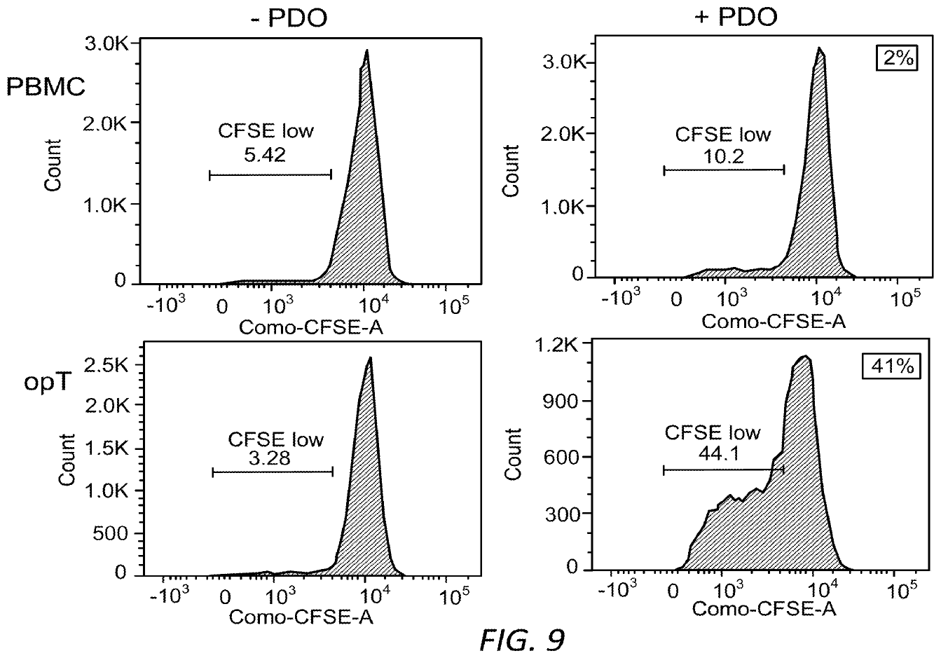

We investigated whether opT and PBMC cells differ in their ability to respond to autologous tumor cells. We labelled PBMC and opT cells with carboxyfluorescein succinimidyl ester (CFSE), an effective method to monitor lymphocyte division. CFSE covalently labels long-lived intracellular molecules with carboxyfluorescein and as the cells divide, daughter cells retaining half the number of carboxyfluorescein-tagged molecules resulting in low-CFSE cell population as analyzed by flow cytometry. This approach provides the ability to monitor up to eight cell divisions. PBMC cells when exposed to tumor cells showed a ˜2.0% increase the population of cells with low CFSE, demonstrating that tumor cells stimulated only a small percentage of cells to enter cell cycle (FIG. 9). In contrast, opT cells showed a >40% increase in CFSE low population of cells (FIG. 9), demonstrating that opT cells show a robust ability to enter cell cycle when exposed to autologous tumor cells validating our ability to generating T cells that respond to tumor cells and hence be used to expand tumor-targeting T cells.

Characterization of opT Cell Phenotype.

To understand the phenotype of opT cells, we assembled a panel of 24 CD markers that can identify different immune phenotypes and a range of T cell activation and memory states. We performed CyToF analysis on PBMC and opT pairs. As expected the PBMC had cells that could be clustered into multiple phenotypic clusters, dominated by T cells (CD4 or CD8) and NKT cells, with some representation by neutrophils, B, myeloid cells. Interestingly, opT cells had restricted diversity with primarily CD8+ T cells, with some representation from CD4+ and NKT cells.

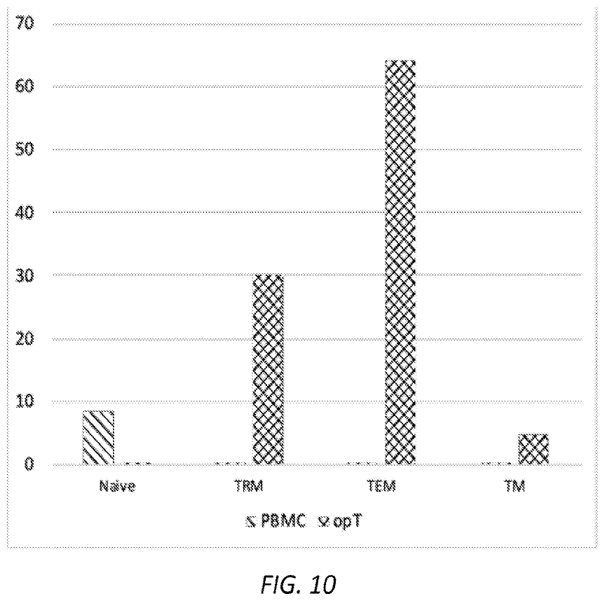

When we specifically analyzed the CD3+ populations, PBMC had 25% CD8 and 75% CD8+ populations with a naïve phenotype, whereas opT cells >95% CD8+ T cells with tissue resident memory or tissue effector memory or transitional memory T cells markers, demonstrating the culture conditions is well suited to retain T cells in different activation and memory states, which makes them ideal for adoptive cell therapy (ACT) application (FIG. 10).

| Phenotype | Markers |
|---|---|
| Naive | CD3+, CD8+, CD45RA+, CCR7+ |
| Tissue-resident memory | CD3+, CD8+, CD103+, CD69+, |
| (TRM) (CD103+, C-kit+) | C-kit+, CD45RA−, CD45RO+, |
| | CDR7− |
| NKG2D+ Tissue Effector | CD3+, CD8+, NKG2D+, CD45RA−, |
| Memory (TEM) | CCR7−, CD28− |
| Transitional memory (TM) | CD3+, CD8+, CD45RA−, CCR7−, |
| | CD28+ |

### Example 4. A Platform for Identifying and Cloning Tumor-Targeting T Cell Receptors

Clonal selection of TCR in opT cells. We next investigated if opT cells are simply an activated population of the T cells in the PBMC culture or the process of opT generation resulted in clonal expansion of T cells that tumor-targeting T cell receptors. that are stimulated by tumor epithelia. More than 150,000 TCR b-chains were sequenced from PBMC and from opT cells. As expected, in PBMC no TCR was represented more than 3.0% demonstrating a polyclonal nature of the population. However, in opT cells one TCR dominated the population representing 79% and two other clones contributing to additional 19 percentage. Thus, three TCRs made up 98% of the diversity in the >150K TCRs analyzed. These observations demonstrate and unexpected and powerful demonstration of clonal expansion occurring in our co-culture conditions, which will serve as a platform for identification and expansion of tumor-targeting T cell clones from peripheral blood of patients with cancer.

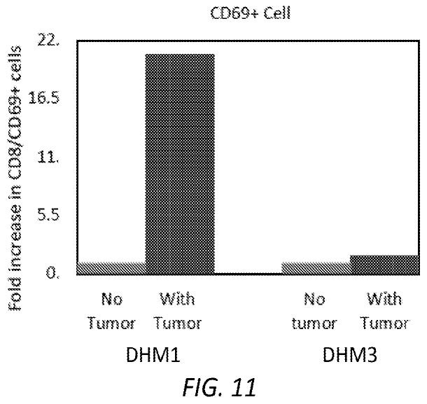

Recombinant T cell receptor selected in opT cells responds to tumor. To determine whether the TCRs present in opT cells retained the ability to recognize tumor when transferred to un-trained T cells, we determined the sequence of the complementary determining region 3 regions (region involved in antigen recognition) from alpha and beta chain of the TCR in opT cells. Two TCRs were selected (DHM1 and DHM2) so that one was enriched in opT cells (DHM1), whereas the other was lost during opT enrichement (DHM3). The CDR3 regions were used to generate a chimeric TCR that comprised of human Valpha and Vbeta chains and mouse constant alpha and beta chains. The chimeric receptor was expressed in SKW-3, a T cell lines that lacks TCR, to investigate if expression of recombinant TCR can initiate for the ability of organoid induce expression T cell activation markers. As shown in FIG. 11, interestingly, only the TCR that was positively selected in opT cells (DHM1), but not ones that were negatively selected-for in opT cells (DHM3), induced expression of T cell activation marker (CD69), demonstrating that TCRs selected in opT cells retain the ability to recognize tumor cells when expressed as a chimeric TCR in untrained T cells.

### REFERENCE

Huang L et al, Ductal pancreatic cancer modeling and drug screening using human pluripotent stem cell- and patient-derived tumor organoids. Nat Med. 2015 November; 21(11):1364-71.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of preparing a co-culture, the method comprising:
obtaining cells from a tumor in a first subject, and preparing a tumor organoid from the cells;
obtaining lymphocytes from the first subject or a second subject, and suspending the lymphocytes in media comprising one, two, or all three of IL-2, IL-15, and IL-21; and
maintaining a co-culture comprising the tumor organoid in the presence of the lymphocytes in media comprising IL-2, IL-15, IL-21 and polyinosinic:polycytidylic acid.

2. The method of claim 1, wherein preparing a tumor organoid comprises:
obtaining a sample comprising tumor tissue;
enzymatically digesting the tissue;
plating single cell suspensions in media comprising Dulbecco's Modified Eagle Media, serum-free supplements, fibroblast growth factors (FGFs), and insulin; and
incubating for 2-3 days.

3. The method of claim 1, wherein the first and second subjects are human.

4. The method of claim 1, wherein the tumor is from a pancreatic, breast, liver, or colon cancer.

5. The method of claim 1, comprising maintaining the co-culture comprising the tumor organoid in the presence of the lymphocytes for at least 3, 4, or 5 days.

6. The method of claim 1, wherein the co-culture is started at a 80:1 to 200:1 ratio of effector cells to target cells, wherein the lymphocytes are effector cells, and wherein the tumor organoids are target cells.

7. The method of claim 1, comprising maintaining the co-culture comprising the tumor organoid in the presence of the lymphocytes for a time sufficient to produce organoid-primed, tumor targeting cytotoxic T cells (opT cells) at least 5 days, and optionally repeating the process two or more times to enrich for opT cells from the co-culture.

8. The method of claim 7, further comprising administering the opT cells to the first or second subject.

9. The method of claim 8, wherein the opT cells are administered to the first subject from whom the tumor cells were obtained.

10. A method of determining sensitivity of a cancer to a test compound, the method comprising:
providing a co-culture prepared by the method of claim 1;
contacting the co-culture with a test compound;
detecting an effect of the test compound on the co-culture by assaying for one or more of proliferation or activity of tumor-killing T cells; proliferation or activity of immune suppressive regulatory T cells, or viability or proliferation of tumor cells; and
identifying a test compound that induces proliferation or activity of tumor-killing T cells, reduces proliferation or activity of immune suppressive regulatory T cells, or directly reduces viability or proliferation of tumor cells as a candidate therapeutic compound.

11. The method of claim 10, wherein the test compound is an immunotherapy.

12. The method of claim 11, wherein the immunotherapy comprises anti-PD1, anti-PDL1 or anti-CTLA4.

13. The method of claim 12, further comprising administering the candidate therapeutic compound to the first subject from whom the tumor cells were obtained.

14. A method of determining tumor neo-antigens, the method comprising:
providing a co-culture prepared using the method of claim 7;
expanding the cells; and
identifying T cell receptors expressed in the cells.
